(19) United States
(12) Patent Application Publication
(10) Pub. No.: US 2006/0085060 A1
(43) Pub. Date: Apr. 20, 2006

(54) Methods and apparatus for coupling an allograft tissue valve and graft

(76) Inventor: Louis A. Campbell, Corona del Mar, CA (US)

Correspondence Address: Medtronic, Inc.;Medtronic Cardiac Surgery Division, 7601 Northland Dr., Brooklyn Park, MN, 55428, US

(21) Appl. No.: 10/966,865

(22) Filed: Oct. 15, 2004

(51) Int. Cl. A61F 2/06; A61F 2/24

(52) U.S. Cl. 623/1.26; 606/153; 623/2.4

(57) ABSTRACT

Improvements to prosthetic heart valves and grafts for human implantation, particularly to methods and apparatus for coupling a prosthetic heart valve with an artificial graft during a surgical procedure to replace a defective heart valve and blood vessel section, e.g., the aortic valve and a section of the ascending aorta, are disclosed. An annular exterior surface of the prosthetic heart valve is fitted within a vascular graft lumen to dispose the vascular graft proximal end overlying the annular exterior surface, and the proximal end of an elongated vascular graft is compressed against the valve annular exterior surface in a manner that inhibits blood leakage between the vascular graft and the prosthetic heart valve.

### FIELD OF THE INVENTION

This invention relates to improvements to prosthetic heart valves and grafts for human implantation, particularly to methods and apparatus for coupling a prosthetic heart valve with an artificial graft during a surgical procedure to replace a defective heart valve and blood vessel section, e.g., the aortic valve and a section of the ascending aorta.

### BACKGROUND OF THE INVENTION

Implantable heart valve prostheses or prosthetic heart valves have been used to replace various diseased or damaged native aortic valves, mitral valves, pulmonic valves and tricuspid valves of the heart. Heart valves are most frequently replaced due to heart disease, congenital defects or infection. The aortic valve controls the blood flow from the left ventricle into the aorta, and the mitral valve controls the flow of blood between the left atrium and the left ventricle. The pulmonary valve controls the blood flow from the right ventricle into the pulmonary artery, and the tricuspid valve controls the flow of blood between the right atrium and the left ventricle. Prosthetic heart valves can be used to replace any of these naturally occurring valves, although repair or replacement of the aortic or mitral valves is most common because they reside in the left heart chambers where the pressure loads are higher and valve failure is more common. Generally, the known prosthetic heart valves are either bioprostheses or mechanical heart valve prostheses.

Modern mechanical heart valve prostheses (hereafter “mechanical valves”) are typically formed of an annular valve seat in a relatively rigid valve body and an occluding disk or pair of leaflets that are movable through a prescribed range of motion between a closed, seated position against the annular valve seat blocking blood flow and an open position allowing blood flow. Such mechanical valves are formed of blood compatible, non-thrombogenic materials, typically currently comprising pyrolytic carbon and titanium. Hinge mechanisms and struts entrap and prescribe the range of motion of the disk or leaflets between the open and closed positions. The MEDTRONIC® Hall® pivoting disk mechanical valve has a pivoting disc occluder and is described in detail in commonly assigned U.S. Pat. Nos. 5,766,240 and 5,948,019. Exemplary bi-leaflet mechanical valves are disclosed in commonly assigned U.S. Pat. Nos. 4,935,030, 6,139,575, and 6,645,244 and in U.S. Pat. Nos. 6,176,877 and 6,217,611.

By their very nature, mechanical valves have metal, pyrolytic carbon, or plastic surfaces exposed to the blood flow, which remain thrombogenic even a long time after their implantation by major surgery. The opening and closing of mechanical valve occluders can damage blood elements and trigger a coagulant cascade. Blood flow disturbances in mechanical valves are also believed to aggravate blood coagulation. Therefore, patients having such mechanical valves can avoid potentially life threatening embolus formation only by taking anti-thrombogenic or anti-coagulant medication on a regular basis.

The bioprostheses (hereafter “tissue valves”) fall into two groups, homografts recovered from human cadavers and xenografts harvested from animal hearts. The most widely used tissue valves include some form of stationary metal or plastic frame or synthetic support, referred to as a “stent”, although so-called “stentless” tissue valves are available. The most common tissue valves are constructed using an intact, multi-leaflet, harvested donor tissue valve, or using separate leaflets cut from bovine (cow) pericardium, for example. The most common intact donor tissue valve used for stented and stentless valves is the porcine (pig) aortic valve, although valves from other animals (e.g., equine or marsupial donors) have been used. Porcine tissue valves include the entire porcine valve in an intact configuration or in some cases, cusps or leaflets from up to three different heart valves excised from pigs then sewn back together. Exemplary tissue valves formed of swine valve leaflets mounted to struts of a stent are those disclosed in U.S. Pat. Nos. 4,680,031, 4,892,541, and 5,032,128 as well as the MEDTRONIC® Hancock II® and Mosaic® stented tissue valves. Some tissue valves, e.g., the MEDTRONIC® Freestyle® stentless aortic root bioprostheses, are formed from treated integral swine valve leaflets and ascending aorta structure.

Tissue valves are preserved by treatment with glutaraldehyde or other chemical preservatives that are rinsed off the exterior surface of the tissue valve before it is sutured to the valvar rim. The blood flow through a preserved porcine tissue is far more physiologic than a mechanical valve, and therefore, the human patient can often avoid taking anti-thrombogenic or anti-coagulant medication. Valve leaflet opening and closing characteristics and blood flow past open tissue leaflets of tissue valves can be superior to those afforded by mechanical valves. However, tissue leaflets can become calcified over time distorting the leaflet shape and ultimately leading to failure of the tissue leaflets to fully close or open.

Proposals have been advanced to form mechanical valves from flexible, anti-thrombogenic, polymeric sheets or fabrics that are resistant to calcification mounted to stents to function like stented or stentless tissue valves as exemplified by U.S. Pat. No. 5,562,729. However, calcification and tear issues of polymeric materials remain to be solved before a polymeric mechanical valve mimicking the function of the leaflets of a tissue valve can be realized.

Such mechanical valves and tissue valves are intended to be sutured to the prepared valvar rim, i.e., the peripheral tissue surrounding the “native annulus” of a natural heart valve orifice after surgical removal of damaged or diseased natural valve structure from the patient's heart. Most modern prosthetic heart valves are typically supplied with a suturing or sewing ring surrounding the valve body or stent that is to be sutured by the surgeon to the valvar rim. Sewing rings typically comprise a fabric strip made of synthetic fiber that is biologically inert and does not deteriorate over time in the body, such as polytetrafluoroethylene (e.g., “Teflon” PTFE) or polyester (e.g., “Dacron” polyester), that is knitted or woven having interstices permeable to tissue ingrowth. The valve body or stent typically has a circular or ring-shaped sidewall shaped to mate with an inner sidewall of the sewing ring, and the sewing ring has an annular outer surface. In some cases, the sewing ring fabric is shaped to extend outward to provide a flattened collar or skirt that can be applied against and sutured to the native tissue annulus, as shown for example in U.S. Pat. Nos. 3,997,923 and 4,680,031. The sewing rings of mechanical heart valves may be rotatable about the valve body as disclosed in the above-referenced '240 patent, for example.

To assure a proper fit, the patient's tissue annulus must be “sized” to indicate the size of the mechanical valve or tissue valve to be implanted in the native annulus. In particular, proper fit of the annular valve body relative to the native annulus of the excised native valve is required. Typically, a set of sizers is supplied by the prosthetic heart valve manufacturer corresponding to the different sizes of available prosthetic heart valves. The surgeon inserts the sizers through the native annulus to determine which corresponding prosthetic heart valve will best fit the native annulus. Thus, it is necessary for the hospital to stock prosthetic heart valves in a range of sizes so that the surgeon can select the appropriately sized prosthetic heart valve during the surgical procedure.

The degeneration of natural heart valves through a disease process is sometimes accompanied by degeneration of blood vessels extending from the heart valve, particularly an aneurysm of the ascending aorta coupled to the aortic valve. Consequently, both the aortic valve and a segment of the ascending aorta may be replaced at the same time. In 1968, Bentall and DeBono described a method for attaching a commercially available graft to a Starr-Edwards mechanical valve for the complete replacement of an aneurysmal aorta and aortic valve. See, “A Technique for Complete Replacement of the Ascending Aorta”, Thorax, (1968), V. 23, pgs. 338-339. In accordance with this technique, the surgeon first sutures the graft to the sewing ring of the mechanical heart valve and then sutures the assembly to the prepared tissue annulus in a conventional manner.

Sewing a graft onto the prosthetic heart valve sewing ring in this manner can be a chalenge, resulting in the possibility of blood leakage through or between the sutured end of the graft and the sewing ring. In addition, the procedure may take an unduly long time, which can cause complications for a patient on cardiac bypass. Moreover, blood leakage through the suture holes typically occurs until the blood coagulates in the holes, and such blood loss is undesirable. See Campbell et al, “DEVELOPMENT OF AN ASCENDING AORTIC VALVED CONDUIT”, Japanese Journal of Artificial Organs, Vol. 21 No. 2, (1992).

Shiley Corp., in conjunction with cardiovascular surgeons, produced a composite mechanical valve and pre-attached graft. A relatively long, tapered fabric section, between 8-12 millimeters long, was disposed between the valve and the constant diameter graft sidewall. It was suggested that the taper would provide a smooth transition between the mechanical valve and the graft to reduce turbulent flow, thereby reducing the risk of embolic events, and to reduce the gradient by eliminating flow separation at a sharp juncture. This hypothesis was disproven by the inventor as explained in the afore-mentioned reference, “DEVELOPMENT OF AN ASCENDING AORTIC VALVED CONDUIT”. Furthermore, the tapered design and fabrication of the graft created problems that made anastomosis of the free ends of the coronary arteries to the sidewall of the graft difficult. The coronary arteries branch from the section of the aorta that typically requires replacement, and the graft and arteries have to be surgically prepared and attached together in an anastomosis. The reduced diameter and method of fabrication of the tapered graft and the design of the sewing ring of the mechanical valve made the anastomosis difficult and time consuming, and blood leakage could occurred about the anastomosed ends of the coronary arteries to the graft. As a result, the long tapered design and fabrication of the graft lost favor and is no longer available.

Improved composite mechanical valves and vascular grafts are disclosed in U.S. Pat. Nos. 5,123,919 and 5,891,195 having a short tapered or non-tapered transition length between the valve and the constant diameter section of the graft, thereby making coronary artery anastomosis less difficult and time consuming. In the '919 and '195 patents, the mechanical valve comprises a rigid circular annular body supporting internal leaflets, a stiffening ring surrounding the annular body, and a sewing ring for attaching the valve to the heart. The stiffening ring also captures a proximal end of the vascular graft between the stiffening ring and the annular body. In the '195 patent, the heart valve has a sewing ring that is substantially impervious to blood flow. In a preferred embodiment, the sewing ring comprises a solid circular silicone washer or insert that supports the sewing ring radially outwardly from the valve and forms a shield to prevent the flow of blood around or through the sewing ring.

Further prosthetic tubular aortic conduits or grafts including at least one graft coupled to a mechanical valve is disclosed in U.S. Pat. No. 6,352,554, particularly with respect to FIG. 5 thereof. The graft is formed having a proximal annular section having a radially expandable sidewall and a distal annular section having a longitudinally extendable sidewall. The mechanical valve is fitted into lumen of the proximal section and is apparently attached to the graft proximal end during manufacture. The radially expandable sidewall mimics the function of the sinuses of Valsalva that are surgically excised during removal of the aorta proximal to the native aortic valve leaflets.

The “mechanical valve/graft combination” disclosed in the '919, '185, and '554 patents has advantages in simplifying, shortening, and making the surgical procedure safer, but the attendant costs to both manufacturers and hospitals are increased. Manufacturers have to supply, and hospitals have to stock a range of such mechanical valve/graft combinations to accommodate the expected native annulus range of a patient population as well as heart valves without attached grafts in a corresponding range of sizes for use in those surgical cases not requiring vessel replacement.

The use of tissue valves continues to increase at the expense of mechanical valves. Consequently, it would also be desirable to provide a “tissue valve/graft combination” to meet the increasing demand for tissue valves despite the attendant supply and stocking costs noted above.

However, there are a number of problems attendant to conceptually providing a tissue valve coupled to a vascular graft. As noted in the above-referenced '195 patent, the development of a graft material, and particularly a porosity of the graft material that both resists blood leakage through it and does not result in neo-intimal peel resulting in emboli, was difficult. In 1991, a mechanical valve/graft combination became available with a medium porosity, graft having fabric pores sealed with collagen or gelatine to inhibit significant blood leakage at the time of surgery. After blood flow is re-established, the sealing material dissolves or is digested and replaced with a fibrin layer that grows into the graft material as the collagen or gelatin is dissolved or digested preventing neo-intimal peel or embolism from part of the fibrin layer peeling off of the graft.

As noted above, tissue valves are preserved by treatment with glutaraldehyde, and they are also stored in sealed containers filled with a glutaraldhyde or other storage solution until removed from the container and washed during a valve replacement surgery. The preservative would attack and wash out the collagen or gelatine coating of a graft connected to the valve and exposed to it during storage. Consequently, it is not possible to supply a viable tissue valve/sealed graft combination.

At the present time, the surgeon that desires to implant a tissue valve and couple it to an aortic graft sutures the proximal end of the graft onto the valve sewing ring at the time of surgery. The surgeon is operating under a significant time pressure because this has to be done while the heart is stopped and the patient is being supported by a heart lung machine. Extended use of the heart lung machine increase the patients risk of emobolism and other complications. Further, the risk of leakage at the point where the surgeon hand sews the graft to the valve is of great concern because the pressure between the inside of the graft and the outside of the graft is fairly high so a patient can lose a significant amount of blood in a short amount of time through a small leak.

### BRIEF SUMMARY OF THE INVENTION

In accordance with the present invention, methods and apparatus are provided for use during a valve replacement procedure for rapidly and securely attaching a prosthetic heart valve to a vascular graft. In accordance with one aspect of the present invention, improved methods and apparatus to couple an elongated vascular graft having a graft sidewall extending between graft proximal and distal ends with a prosthetic heart valve for replacement of a section of blood vessel and a native heart valve are provided.

The methods of the present invention include the steps of fitting an annular exterior surface of the prosthetic heart valve within a vascular graft lumen to dispose the vascular graft proximal end overlying the annular exterior surface, and compressing the proximal end of an elongated vascular graft against the valve annular exterior surface in a manner that inhibits blood leakage between the vascular graft and annular exterior surface of the heart valve where attached to the prosthetic heart valve.

The annular surface is preferably an exterior surface of the sewing ring of a mechanical valve or a tissue valve or a valve body surface adjacent the sewing ring. An annular proximal section of the vascular graft is clamped against the sewing ring or valve body in a region that does not interfere with suturing the sewing ring to the patient's valvar rim and with valve leaflet function.

The methods of the present invention are advantageously performed employing stock tissue valves or mechanical valves that can be used alone or in conjunction with the vascular graft. Therefore, it is not necessary to supply and stock separate mechanical or tissue valves intended to be coupled with the elongated vascular graft, and the stock tissue valves or mechanical valves, and the elongated vascular graft may be advantageously usable separately.

The apparatus of the present invention comprises clamping elements that are applied around the proximal section of the elongated vascular graft that is itself applied against the valve annular exterior surface. The clamping elements are advantageously either fixed to the graft proximal end when the vascular graft is fabricated or are separately provided with the vascular graft.

Advantageously, the clamping elements are relatively inexpensive, and more than one can be supplied for use alone or in combination as the surgeon considers appropriate. Thus, the costs of providing and acquiring the capability of coupling a vascular graft to a stock prosthetic heart valve are kept low.

Moreover, the methods and apparatus of the present invention advantageously enable the rapid and secure coupling of a chemically preserved and sterile tissue valve with a sterile vascular graft in the sterile operating theater with minimal degradation of vascular graft coatings that acutely inhibit blood leakage through the fabric forming the graft sidewall. The tissue valve is first rinsed of the chemical preservative and is then coupled with the graft proximal end.

The present invention may be employed with a relatively constant diameter vascular graft or a vascular graft having an expanded graft proximal section of the type disclosed in the above-referenced '554 patent or a vascular graft having a tapered graft proximal section.

The present invention has the potential to enable rapid, secure attachment of a stock tissue or mechanical valve selected to meet the patient's need with a vascular graft during the surgical procedure to minimize trauma to the patient and not increase manufacturing and inventory costs.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic representation of the replacement of a dysfunctional aortic heart valve and a section of the ascending aorta with a prosthetic heart valve and a vascular graft coupled together in accordance with the present invention;

FIG. 2 is a side plan view of an elongated vascular graft that may be employed in the procedure depicted in FIG. 1 employing one of the clamping techniques of the present invention;

FIG. 3 is a side plan view of an elongated vascular graft and a tissue valve positioned in relation to a compression ring with an expanded inner diameter in preparation for disposing a proximal section of the vascular graft around an annular exterior surface of the valve sewing ring and disposition of the compression ring around the proximal section of the vascular graft;

FIG. 4 is a side view in partial cross-section of the proximal section of the vascular graft of FIG. 3 disposed around and against the annular exterior surface of the valve sewing ring and disposition of the compression ring with an expanded inner diameter surrounding the proximal section of the vascular graft;

FIG. 5 is a side view in partial cross-section of the proximal section of the vascular graft of FIG. 3 disposed around and against the annular exterior surface of the valve sewing ring and the compression ring bearing against the proximal section of the vascular graft upon release of the expanded inner diameter, thereby coupling the vascular graft proximal end to the tissue valve;

FIG. 6 is a side plan view of an elongated vascular graft and a tissue valve positioned for disposing a proximal section of the vascular graft around an annular exterior surface of the valve sewing ring, wherein a resilient compression ring is supported within a proximal section of the vascular graft forming a proximal rim;

FIG. 7 is a side view in partial cross-section of the proximal section of the vascular graft of FIG. 6 disposed around and against the annular exterior surface of the valve sewing ring with the compression ring expanded against and surrounding the proximal section of the vascular graft;

FIG. 8 is a side view in partial cross-section of the proximal section of the vascular graft of FIG. 6 disposed around and against the annular exterior surface of the valve sewing ring with the compression ring expanded against and surrounding the proximal section of the vascular graft and further depicting redundant or alternative securing barbs and a drawstring suture;

FIG. 9 is a plan view of an alternative compression ring comprising a garter spring having a relaxed first inner diameter that can be expanded to an expanded second inner diameter;

FIG. 10 is a side view in partial cross-section of the proximal section of the vascular graft of FIG. 2 disposed around and against the annular exterior surface of the valve sewing ring with the garter spring compression ring expanded to the second inner diameter against and surrounding a proximal section of the vascular graft;

FIG. 11 is an expanded section view of FIG. 10 showing the garter spring compression ring forcing the annular proximal section of the vascular graft against the annular exterior surface of the tissue valve and depicting optional application of a drawstring suture;

FIG. 12 is a plan view of an alternative compression ring comprising a split ring having a relaxed first inner diameter and an exterior channel adapted to receive a drawstring suture;

FIG. 13 is a side view of the compression ring of FIG. 12 with an expanded second inner diameter when placed over the exterior surface of the annular proximal section of the vascular graft of FIG. 2 that is in turn applied over the exterior surface of the prosthetic heart valve of FIG. 1 to apply clamping force thereto;

FIG. 14 is an expanded section view of the compression ring of FIG. 12 applied around and over the annular proximal section of the graft that is in turn applied around the annular exterior surface of the tissue valve forcing them together and further depicting a drawstring applied to force the annular proximal section of the graft into the exterior channel and to maintain the second inner diameter of the compression ring;

FIG. 15 is a plan view of a further alternative compression ring comprising a split ring with loops at ring free ends and a locking pin inserted into the loops to expand the ring inner diameter to an expanded first inner diameter to enable insertion of the annular proximal section of the graft overlying the annular exterior surface of the heart valve;

FIG. 16 is a side view of the compression ring of FIG. 15 with the locking pin in place maintaining the expanded first inner diameter;

FIG. 17 is a side view of the compression ring of FIG. 15 with the locking pin removed allowing compression ring to return to a relaxed second inner diameter that applies clamping force of the annular proximal section of the graft against the annular exterior surface of the heart valve;

FIG. 18 is a side plan view of an elongated vascular graft and a mechanical valve positioned in relation to a compression ring with an expanded inner diameter in preparation for disposing a proximal section of the vascular graft around an annular exterior surface of the valve sewing ring and disposition of the compression ring around the proximal section of the vascular graft;

FIG. 19 is a side view in partial cross-section of the proximal section of the vascular graft of FIG. 18 disposed around and against the annular exterior surface of the valve sewing ring and disposition of the compression ring with an expanded inner diameter surrounding the proximal section of the vascular graft;

FIG. 20 is a side view in partial cross-section of the proximal section of the vascular graft of FIG. 18 disposed around and against the annular exterior surface of the valve sewing ring and the compression ring bearing against the proximal section of the vascular graft upon release of the expanded inner diameter, thereby coupling the vascular graft proximal end to the mechanical valve;

FIG. 21 is a side view in partial cross-section of the proximal section of a vascular graft disposed around and against the annular exterior surface of a valve sewing ring and both a compression ring and a drawstring bearing against the proximal section of the vascular graft coupling the vascular graft proximal end to the mechanical valve; and

FIG. 22 is a side view in partial cross-section of the proximal section of a vascular graft disposed around and against the annular exterior surface of a valve sewing ring of a tissue valve, wherein the tissue valve stents are formed having an annular retention channel receiving the compression ring.

### DETAILED DESCRIPTION OF THE INVENTION

In the following detailed description, references are made to illustrative embodiments of methods and apparatus for carrying out the invention. It is understood that other embodiments can be utilized without departing from the scope of the invention. Preferred methods and apparatus are described for rapidly and securely coupling a tissue valve or a mechanical valve with a graft during the replacement of a native heart valve and portion of the aorta in a heart. It will be understood that the coupling methods and compression rings that are depicted in the drawings in relation to either a tissue valve or a mechanical valve may be used for both tissue and mechanical valves unless otherwise explicitly stated. The particular manner of fabrication of the individual components of the tissue valves and mechanical valves are not of importance to the present invention.

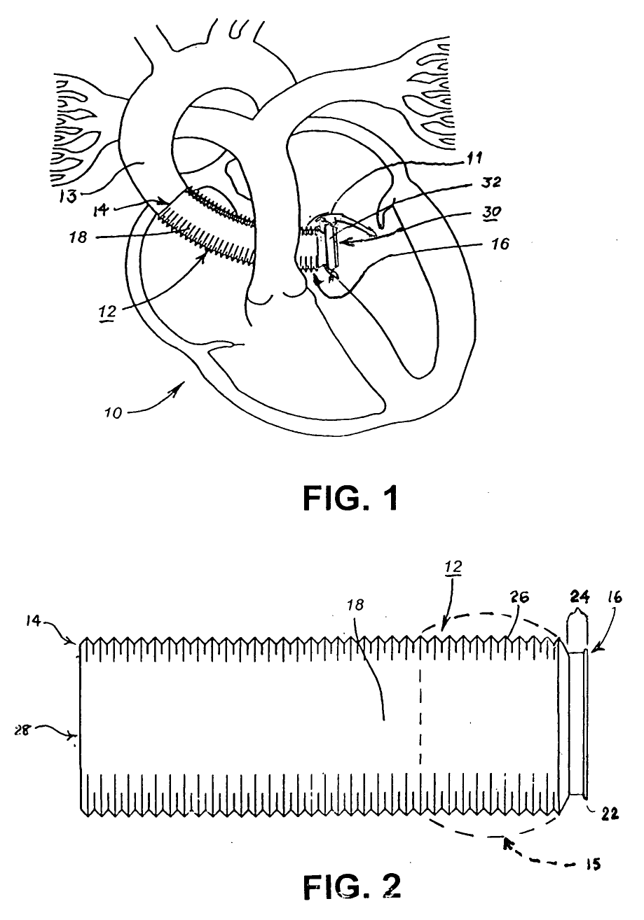

FIG. 1, similar to FIG. 1 of the above-referenced '195 patent, is a partial cross-section, schematic view of a human heart 10 showing the surgical replacement of the aortic valve and a section of the ascending aorta 13. The coronary arteries (not shown) are excised from the section of the ascending aorta that is removed. The aortic valve is removed, and the valvar rim 11 is prepared. The valve annulus is sized, and a prosthetic heart valve 30 of appropriate size is selected. An elongated vascular graft 12 extending from a graft distal end 14 to a graft proximal end 16 is also selected. The selected prosthetic heart valve 30 is coupled to the graft proximal end 16 in accordance with the present invention. The sewing ring 32 of the prosthetic heart valve 30 is sutured to the prepared valvar rim 11 where the aortic valve was removed. The graft distal end 14 is sutured to the severed end of the ascending aorta 13 superior to the excise portion. It will be understood that the severed ends of the coronary arteries (not shown) would be coupled by anastomosis to the sidewall 18 of the graft 12 between the heart valve 30 and the ascending aorta 13.

The vascular graft 18, also shown in FIG. 2, comprises a tubular structure having a corrugated or pleated sidewall 26 extending between proximal and distal graft ends 16 and 14 formed of a biocompatible fabric of the type described above. The fabric sidewall 18 is coated or treated with a material, e.g., collagen or gelatine, which inhibits blood leakage in the acute post-operative stage as described above. The sidewall 18 of graft 12 may comprise the sidewall employed in the Gelweave aortic graft sold by Vascutek Ltd, Inchinnan, UK or the sidewall employed in the Hemashield aortic graft sold by Boston Scientific Corporation, Natick, Mass.

The sidewall 18 is formed having a short, annular proximal section 24 having a rim 22 at the graft proximal end 16 that is adapted to be coupled to the heart valve 30 as shown in FIG. 1. As disclosed in the above-referenced '195 patent, a taper can be formed between the annular proximal section 24 and the remaining distal section of the sidewall 18 by removing small, triangular, fabric wall sections and sewing the resulting edges together. Usually, four such sewn features spaced around the circumference of the sidewall 18. The tapered section is extremely short, in the range of two mm to four mm, and the sewn edges do not extend into a region where the coronary arteries would be attached. The ostia of the coronary arteries, therefore, can be attached into the graft sidewall 18 immediately downstream from the prosthetic heart valve 30. The circumference of the graft sidewall 18 at the point of attachment of the coronary arteries is relatively large, and the large circumference permits the arteries to be attached without stretching.

Various embodiments of the vascular graft 12 are schematically depicted in the figures that can be employed with various embodiments of compression rings to couple the proximal sections of the vascular graft embodiments against an annular exterior surface of the prosthetic heart valve, particularly the sewing ring surrounding the valve body. The annular proximal sections of the various embodiments of the vascular graft is somewhat resilient in that it may be stretched to snugly fit over the annular exterior surface and may be rolled back on itself or over a retaining member. In certain embodiments, the proximal end 16 is formed with a ring-shaped, resilient retaining member, like an O-ring or a garter spring that is sized to fit a range of valve body diameters.

The vascular graft 12 may also preferably include an annular section 15 having a radially expandable sidewall (shown schematically in broken lines in FIG. 2) as disclosed in the above-referenced '554 patent to mimic the function of the sinuses of Valsalva that are surgically excised during removal of the aorta proximal to the native aortic valve leaflets.

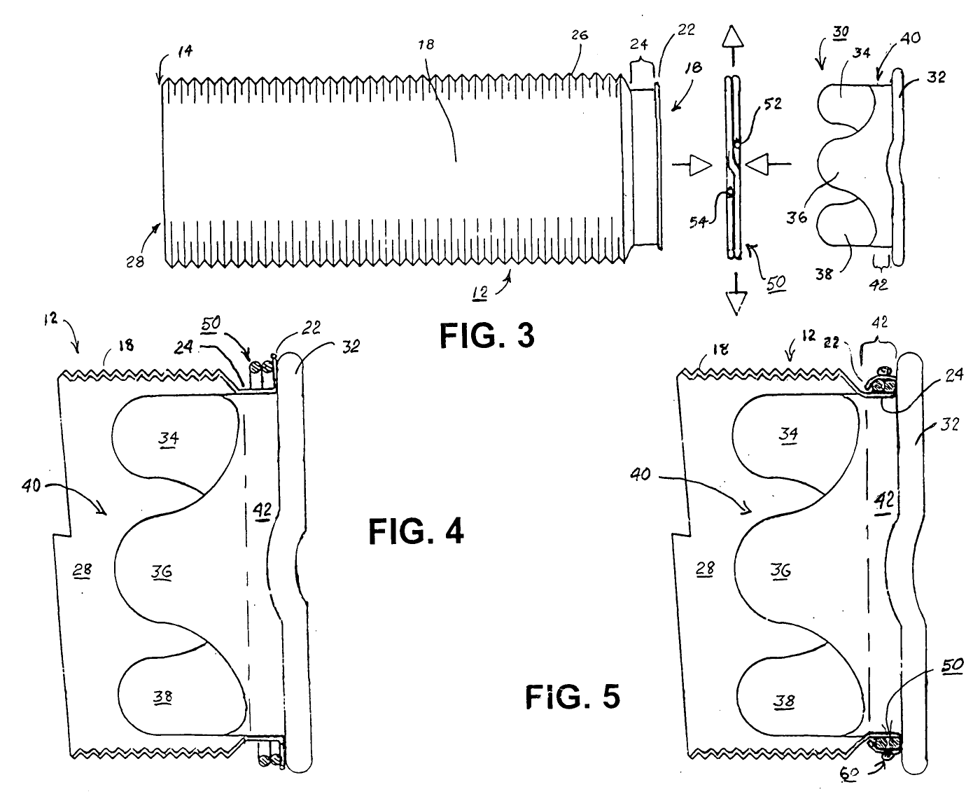

Turning to the embodiment depicted in FIGS. 3-5, the prosthetic heart valve comprises a tri-leaflet tissue valve 30 of the type described in the above-referenced '031 patent, for example. Generally speaking, the tissue valve 30 comprises a sewing ring 32 mounted to an annular, fabric covered stent 40 having three stent struts 34, 36, 38 extending away from the sewing ring 32. The tissue leaflets supported by stent struts 34, 36 and 38 are not show in the figures for convenience of illustrating the aspects and embodiments of the present invention. The stent 40 and tissue leaflets can take any of the forms known in the art such as those described in the background of the invention.

The sewing ring fabric comprises a fabric strip made of synthetic fiber of the types described above of a mesh knit or weave having interstices permeable to tissue ingrowth. The stent 40 typically has a circular or ring-shaped sidewall shaped to mate with an inner sidewall of the sewing ring 32, and the sewing ring 32 has an annular outer surface. The sewing ring fabric is shaped to extend outward to provide a flattened collar or skirt that can be applied against and sutured to the native tissue annulus, as shown for example in U.S. Pat. Nos. 3,997,923 and 4,680,031. A section of the sewing ring 32 may extend over the stent 40 toward the struts 32 to present an annular exterior surface 42 adjacent the outwardly extending skirt of the sewing ring 32.

The retaining member comprises a split ring 50, like a key ring, formed of bio-compatible metal or plastic having first and second ring ends that are spaced apart. The split ring 50 is formed to have an unrestrained first ring lumen diameter that can be increased to a larger second ring lumen diameter through the use of a tool of the type disclosed in U.S. Pat. No. 6,716,243, for example, to fit the split ring 50 over the annular proximal section 24 of the vascular graft 12 that is in turn fitted overlying the annular exterior surface 42 as shown in FIG. 4. The annular proximal section 24 of the vascular graft 12 can be stretched somewhat at the rim 22 as shown in FIG. 4. Thus, the annular proximal section 24 of the vascular graft 12 is disposed around and against the annular exterior surface 42 of the sewing ring 32, and the expanded compression ring 50 is disposed surrounding the annular proximal section 24 of the vascular graft 12 in FIG. 4.

In FIG. 5, the force expanding the ring lumen diameter of the compression ring 50 is released, and the compression ring 50 contracts to attempt to restore the first ring lumen diameter. The ring lumen diameter may be at the first ring lumen diameter or at a somewhat larger ring lumen diameter depending on the difference between the first ring lumen diameter, the diameter of the annular exterior surface, and the rigidity and bulk of the annular proximal section 24 and the underlying sewing ring 32 and/or stent 40. The released compression ring 50 then applies compression force against the proximal section 24 of the vascular graft 12 pressing it against the annular exterior surface 42, thereby coupling the vascular graft proximal end 16 to the tissue valve 30. The flexible sewing ring 32 may be compressed inward somewhat depending on the applied compression force. The rim 22 is wrapped over the outer surface of the split ring 50 as shown in FIG. 5. Optionally, a drawstring suture 60 may be wrapped around the split ring 50 enclosed within the folded over annular proximal section 24 and tied off.

The split ring 60 can be employed in this manner to alternatively couple the graft proximal end 16 to an annular exterior surface of a mechanical valve. The sprit ring 60 may take other forms than depicted in FIGS. 3-5, e.g., a split ring with tapered mating surfaces of the type disclosed in the above-referenced '243 patent.

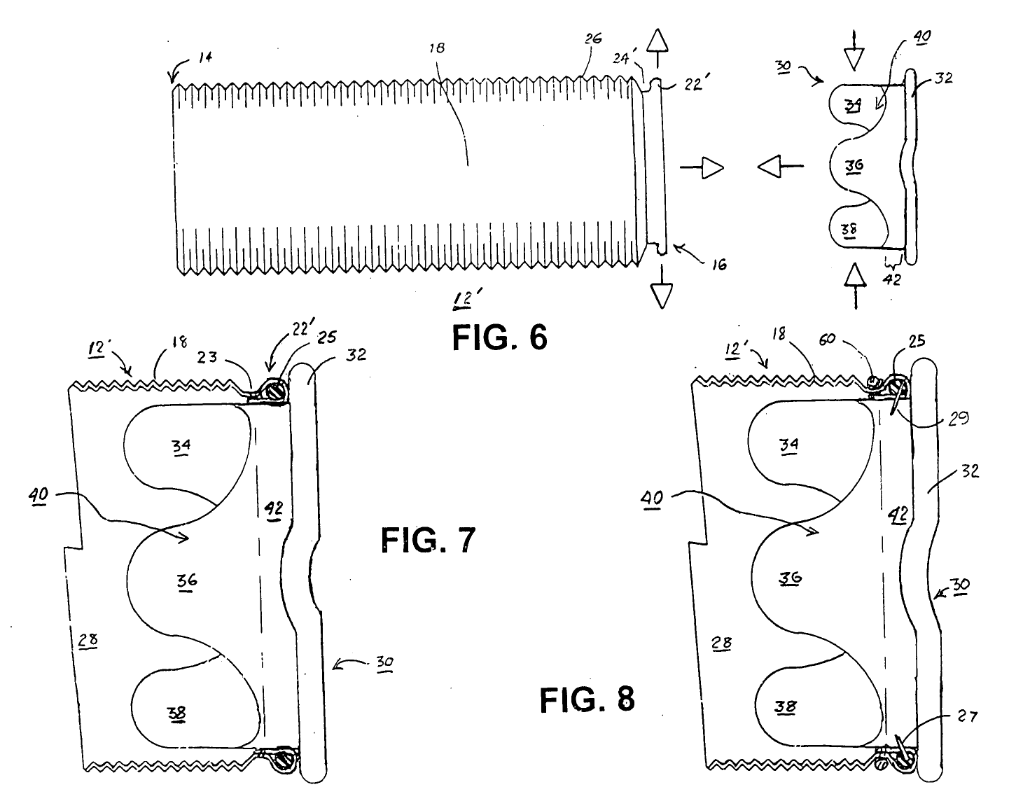

A further embodiment of the invention is depicted in FIGS. 6-8, wherein a modified proximal rim 22′ is provided at the graft proximal end 16 of the modified graft 12′. The modified proximal rim 22′ shown in FIGS. 7 and 8 comprises a resilient compression ring 25 comprising an elastomeric O-ring or a split ring or a garter spring, that is sewn into the fabric 18 by stitches 23 passing through the annular proximal section 24′. The modified proximal rim 22′ has an unrestrained rim or compression ring first ring lumen diameter that is sized to be smaller than the outer diameter of the annular exterior surface 42 of the tissue valve 30. The compression ring lumen diameter can be increased to a second ring lumen diameter by radial stretching forces applied as shown in FIG. 6 as the valve cusps and stents 34, 36, 38 are advanced into the graft lumen 28. The stents 34, 36, 38 of such a typical tissue valve 30 can be flexed inward toward one another as shown in FIG. 6 to facilitate advancement of the valve cusps and stents 34, 36, 38 into the graft lumen 28.

The force expanding the ring lumen diameter of the compression ring 25 to the expanded second ring lumen diameter is released, and the first ring lumen diameter tends to be restored to a degree dependent on the difference between the first ring lumen diameter, the diameter of the annular exterior surface, and the rigidity and bulk of the annular proximal section 24 and the underlying sewing ring 32 and/or stent 40. The flexible sewing ring 32 may be compressed inward somewhat depending on the applied compression force. The compression ring 25 then applies compression force against the proximal section 24 of the vascular graft 12 pressing it against the annular exterior surface 42, thereby coupling the vascular graft proximal end 16 to the tissue valve 30.

The compression ring 25 and rim 22 are depicted fully seated against the annular exterior surface 42 of the sewing ring 32 (or stent 40) in FIGS. 7 and 8. A drawstring suture 60 may be applied as shown in FIG. 8 to provide a redundant retention force. In addition, or alternatively, the rim 22′ may be formed as shown in FIG. 8 with a plurality of minute teeth 27, 29 that extend inward of the compression ring lumen. The teeth 27, 29 may comprise barbs or prongs or Velcro-like hooks that bend inward during advancement of the valve cusps and stents 34, 36, 38 into the graft lumen 28 and that bite into the sewing ring fabric or the fabric covering the stent 40 when the compression ring 25 is fully seated to grip and resist withdrawal of the tissue valve 30 from the graft lumen 28. The teeth 27, 29 may be formed integrally with the resilient compression ring 25 or formed extending from a separate band fitting within the rim opening and may pass through fabric pores a sufficient distance to grip the fabric.

The modified vascular graft 12′ can be employed in this manner to alternatively couple the graft proximal end 16 to an annular exterior surface of a mechanical valve. The exterior sidewalls of mechanical and tissue valves may exhibit annular grooves of the type depicted in the stent of the tissue valve of FIG. 22 described further below, that would further strengthen the engagement of the rim 22′ against the annular exterior surface 42.

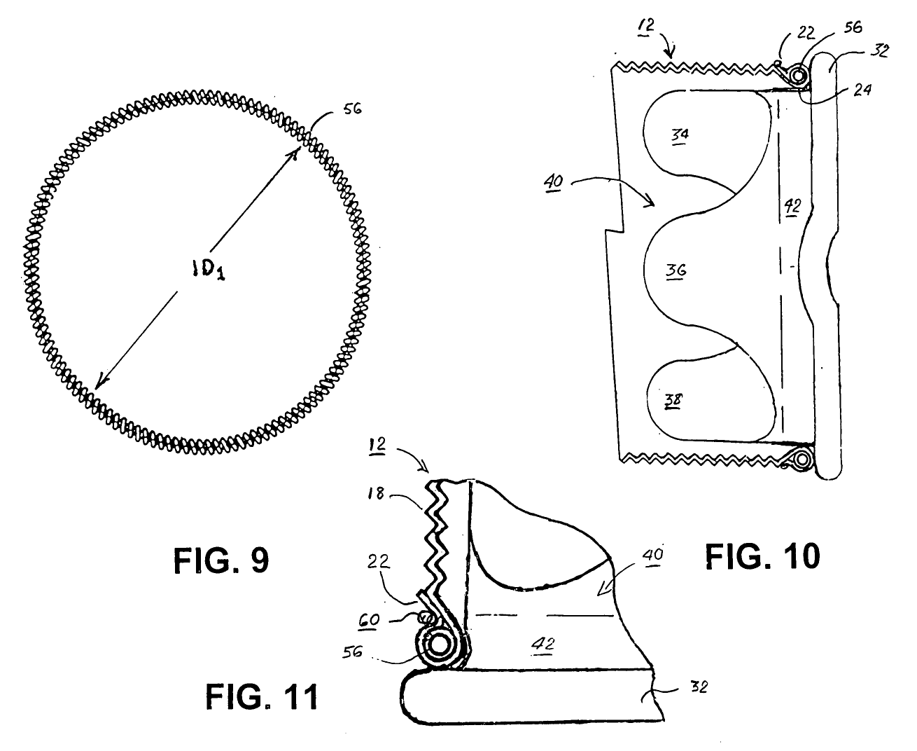

The alternative use of a garter spring 56 formed of bio-compatible metal or plastic as a compression ring is depicted in FIGS. 9-11. The garter spring 56 has a relaxed first ring lumen diameter ID₁ that can be expanded to an expanded second ring lumen diameter ID₂ while the garter spring is applied over the annular exterior surface 42 surrounding the graft proximal section 24.

The proximal section 24 of the vascular graft 12 is disposed around and against the annular exterior surface 42 of the sewing ring 32 (or stent 40) with the garter spring 56 expanded to the second inner diameter ID₂ against and surrounding the graft proximal section 24. The first ring lumen diameter ID₁ tends to be restored to a degree dependent on the difference between the first ring lumen diameter ID₁, the diameter of the annular exterior surface, and the rigidity and bulk of the annular proximal section 24 and the underlying sewing ring 32 and/or stent 40. The flexible sewing ring 32 may be compressed inward somewhat depending on the applied compression force. The rim 22 is wrapped over the outer surface of the garter spring 56 as shown in FIGS. 10 and 11. Optionally, a drawstring suture 60 may be wrapped around the garter spring 56 enclosed within the folded over annular proximal section 24 and tied off.

The garter spring 56 can be employed in this manner to alternatively couple the graft proximal end 16 to an annular exterior surface of a mechanical valve. The exterior sidewalls of mechanical and tissue valves may exhibit annular grooves of the type depicted in the stent of tissue valve of FIG. 22 described further below, that would further enhance the retention of the garter spring 56 against the annular exterior surface 42.

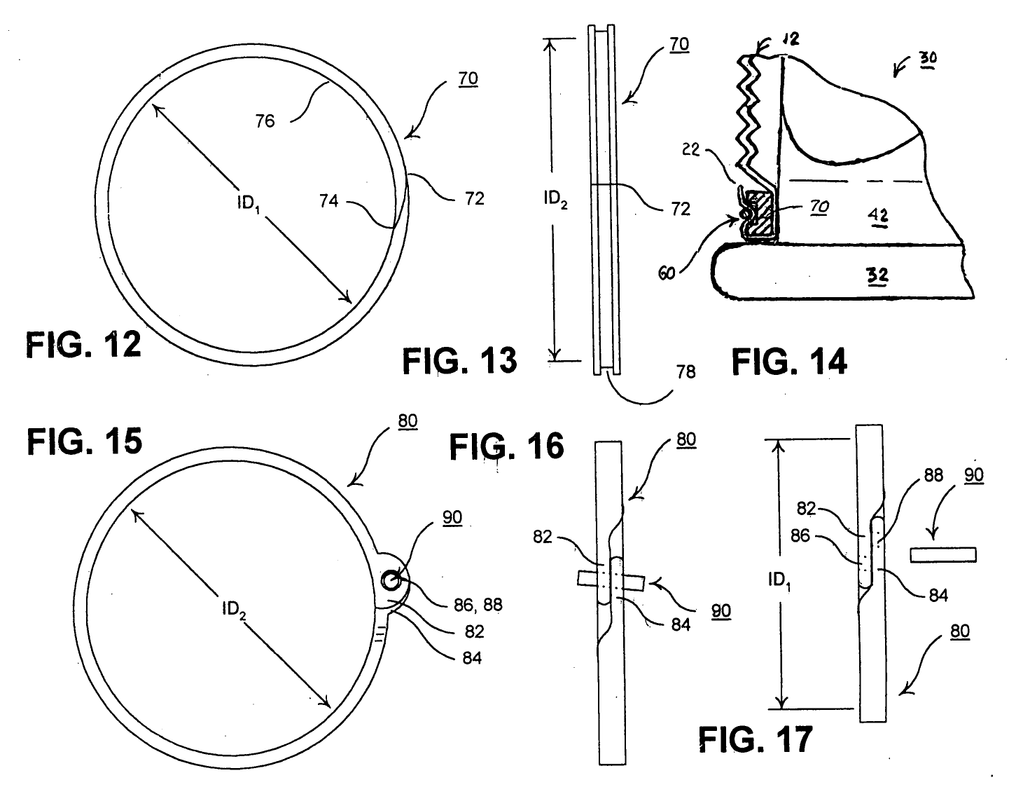

A still further embodiment of a compression ring is depicted in FIGS. 12-14 comprising a split ring 70 formed of bio-compatible metal or plastic having overlapping first and second ring ends 72 and 74 and an outer surface annular channel 78. The split ring 70 has a relaxed first ring lumen diameter ID₁ that can be expanded to an expanded second ring lumen diameter ID₂. The proximal section 24 of the vascular graft 12 is disposed around and against the annular exterior surface 42 of the sewing ring 32 (or stent 40) with the split ring 70 expanded to the second inner diameter ID₂, and the split ring surface 76 is applied against and surrounding the graft proximal section 24. The first ring lumen diameter ID₁ tends to be restored to a degree dependent on the difference between the first ring lumen diameter ID₁, the diameter of the annular exterior surface, and the rigidity and bulk of the annular proximal section 24 and the underlying sewing ring 32 and/or stent 40. The flexible sewing ring 32 may be compressed inward somewhat depending on the applied compression force. The rim 22 is wrapped over the outer surface of the as shown in FIG. 14. A drawstring suture 60 can then be wrapped around the annular channel 78 of the split ring 70 enclosed within the folded over annular proximal section 24 and tied off as shown in FIG. 14.

The split ring 70 can be employed in this manner to alternatively couple the graft proximal end 16 to an annular exterior surface of a mechanical valve. The exterior sidewalls of mechanical and tissue valves may exhibit annular grooves of the type depicted in the stent of the tissue valve of FIG. 22 described further below, that would further strengthen the retention of the split ring 70 against the annular exterior surface 42.

Yet another embodiment of a compression ring is depicted in FIGS. 15-20 comprising a split ring 80 formed of bio-compatible metal or plastic having side-by-side overlapping ring free ends 82 and 84. Although a single ring loop is shown, multiple loops may be employed to obtain the optimum amount of compressive force to provide a reliable seal between the graft and the valve. Bores 86 and 88 extend through the respective ring free ends 82 and 84 that can be forced into alignment to receive a locking pin 90. The pin free ends 82 and 84 and the bores 86 and 88 extending therethrough may take a number of shapes that can be characterized as loops. The split ring 80 has a relaxed first ring diameter ID₁ depicted in FIG. 17 that can be expanded to an expanded second ring diameter ID₂ as shown in FIG. 15. The expanded second ring diameter ID₂ is maintained by the locking pin 90 fitted though the aligned pin receptacles or bores 86 and 88 as shown in FIG. 16.

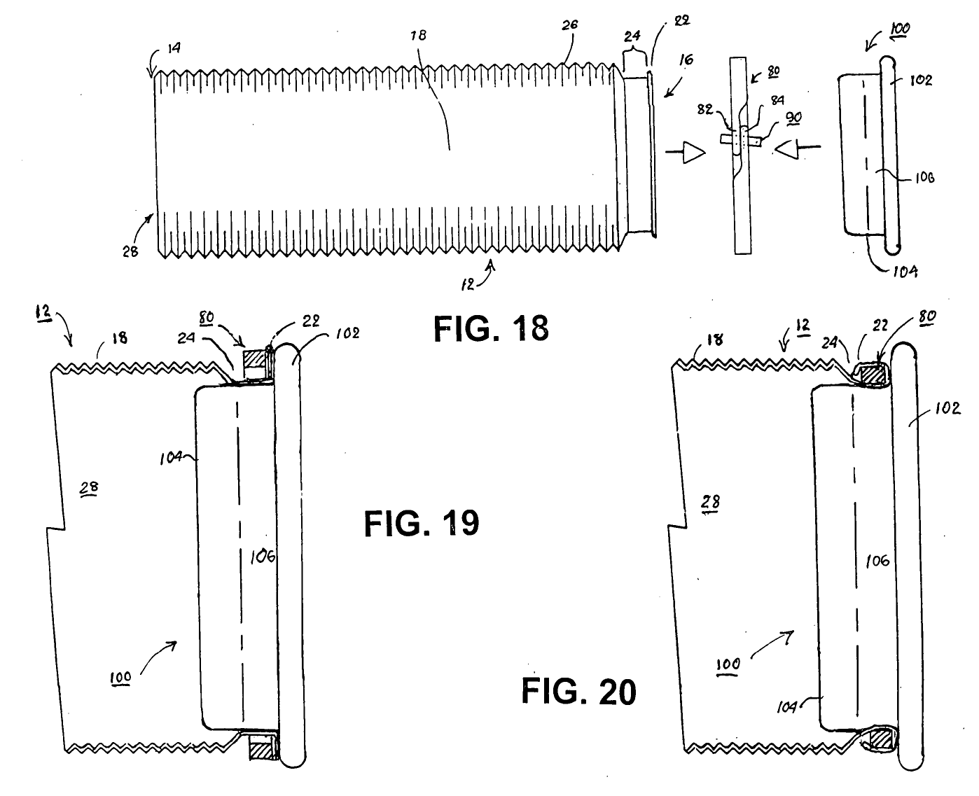

The split ring 80, with the locking pin 90 in place, is depicted in FIG. 18 in operative relation to vascular graft 12 and a mechanical valve 100. The mechanical valve 100 may take any of the forms disclosed above or in the prior art and comprises an annular sewing ring 102 surrounding all or part of the annular outer surface of the valve body 104. The sewing ring 102 may or may not have an outwardly extending flange as depicted in FIGS. 18-20 and may or may not be rotatable with regard to the valve body 104. For convenience, the valve leaflet or leaflets are not shown in FIGS. 18-20.

The split ring 80, with the locking pin 90 in place, is depicted in FIG. 19 overlying the annular proximal section 24 of the vascular graft 12 and the annular outer surface 106 of the mechanical valve 100. The pin 90 is withdrawn in FIG. 20, and the relaxed first ring diameter ID₁ tends to be restored applying compressive force against and surrounding the graft proximal section 24 trapping it against the annular outer surface 106. The first ring lumen diameter ID₁ tends to be restored to a degree dependent on the difference between the first ring lumen diameter ID₁, the diameter of the annular exterior surface, and the rigidity and bulk of the annular proximal section 24 and the underlying sewing ring 106. The flexible sewing ring 102 may be compressed inward somewhat depending on the applied compression force. The rim 22 is wrapped over the outer surface of the split ring 80 as shown in FIG. 20. A drawstring suture can then be wrapped around the split ring 80 enclosed within the folded over annular proximal section 24 and tied off as shown in FIG. 14.

The split ring 80 can be employed in this manner to alternatively couple the graft proximal end 16 to an annular exterior surface 42 of a tissue valve 30. The exterior sidewalls of mechanical and tissue valves may exhibit annular grooves of the type depicted in the stent of the tissue valve 30′ of FIG. 22 described further below, that would further strengthen the retention of the split ring 80 against the annular exterior surface 106.

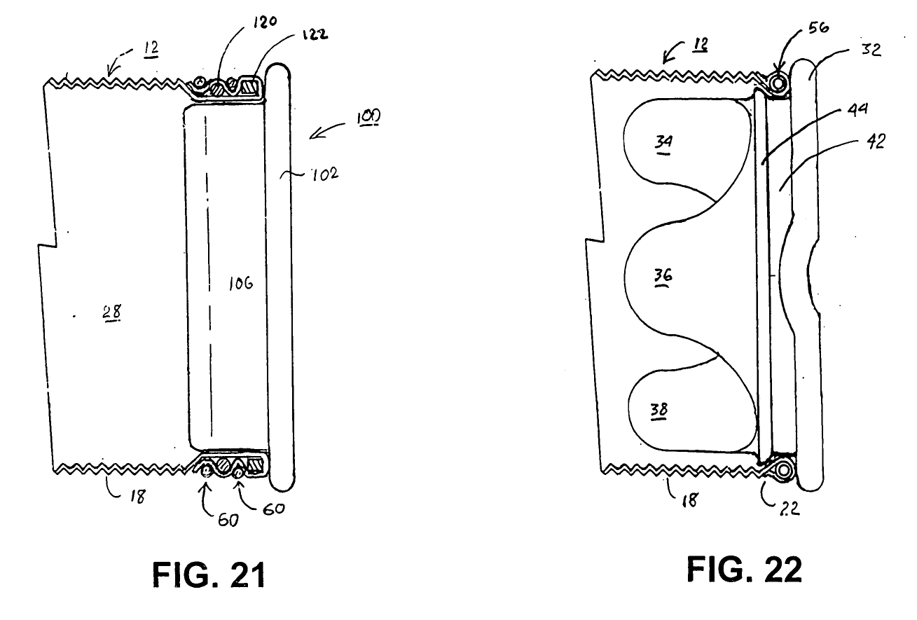

FIG. 21 is a side view in partial cross-section of the proximal section of a vascular graft 12 disposed around and against the annular exterior surface 106 of a valve sewing ring 102 with one or more solid ring 120, 122 and a drawstring(s) 60 bearing against the proximal section of the vascular graft 12 coupling the vascular graft proximal end to the mechanical valve 100. The solid rings 120, 122 are not necessarily expandable, and the drawstring sutures(s) 60 apply the compressive force alongside a single solid ring 120 or 122 or between a pair of solid rings 120 and 122.

FIG. 22 is a side view in partial cross-section of the proximal section of a vascular graft 12 disposed around and against the annular exterior surface 42 of a valve sewing ring 32 of a tissue valve 30, wherein the tissue valve stents 34, 36, 38 are formed having an annular retention channel at annular outer surface 42 created by an outwardly extending band 44. The annular retention channel thereby receives the compression ring, in this case the above-described garter spring 56, for example. Placing the compression ring in the annular channel of such a tissue valve is made easier because the stents 34, 36, 38 are somewhat flexible and can be bent inward as described above with respect to FIG. 6. It will be understood that FIG. 22 is representative of any of the mechanical or tissue valves that are formed with an annular retention channel within or alongside the suture ring that the compression ring can be fitted into in accordance with the teachings of the present invention. The sold rings 120 or 122 may comprise expandable or non-expandable slip rings that are sized to slip over the graft proximal end overlying the annular exterior surface. The drawstring suture(s) 60 applies compression force against the vascular graft proximal end adjacent the slip ring 120, 122 and at least a portion of the annular exterior surface when the drawstring suture(s) 60 is drawn tight.

The above-described and depicted compression rings may be shaped to have non-uniform inner ring diameters, whereby only portions of the inner ring surface applies compression force to clamp the graft proximal end to the mechanical or tissue valve.

All patents and publications referenced herein are hereby incorporated by reference in their entireties.

Certain of the above-described structures, functions and operations of the above-described preferred embodiments are not necessary to practice the present invention and are included in the description simply for completeness of an exemplary embodiment or embodiments.

In addition, it will be understood that specifically described structures, functions and operations set forth in the above-referenced patents can be practiced in conjunction with the present invention, but they are not essential to its practice. It is therefore to be understood, that within the scope of the appended claims, the invention may be practiced otherwise than as specifically described without actually departing from the spirit and scope of the present invention.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

1. A method of coupling an elongated vascular graft having a graft sidewall extending between graft proximal and distal ends with a prosthetic heart valve for replacement of a section of blood vessel and a native heart valve comprising:
fitting an annular exterior surface of the prosthetic heart valve within a vascular graft lumen to dispose the vascular graft proximal end overlying the annular exterior surface; and
clamping the graft proximal end against at least a portion of the annular exterior surface without perforating the vascular graft to couple the vascular graft with the prosthetic heart during chronic implantation and to inhibit blood leakage through the proximal end of an elongated vascular graft.

2. The method of claim 1, further comprising:
providing an expandable compression ring having a ring lumen adapted to be expanded from a first ring lumen diameter sized with respect to the annular exterior surface to a second ring lumen diameter; and
wherein:
the fitting step further comprises:
expanding the ring lumen to the second ring lumen diameter; and
fitting the graft proximal end overlying the annular exterior surface within the expanded ring lumen; and
the clamping step further comprises halting the expanding to allow the ring lumen to contract toward the first ring lumen diameter, thereby applying compression force of the compression ring against the vascular graft proximal end and at least a portion of the annular exterior surface.

3. The method of claim 2, wherein the prosthetic heart valve comprises a valve body supporting at least one occluding member and an annular, resilient sewing ring disposed about the valve body and the annular exterior surface has an annular surface diameter.

4. The method of claim 3, wherein the providing step comprises providing the expandable compression ring having a first ring lumen diameter sized with respect to the annular surface diameter to provide an interference fit compressing the vascular graft proximal end against the resilient sewing ring.

5. The method of claim 2, wherein the prosthetic heart valve comprises one of a tissue valve and a mechanical valve.

6. The method of claim 2, wherein:
the providing step comprises providing a split ring formed of a biocompatible metal and having an annular channel extending around the circumference of the split ring; and
the clamping step comprises extending a drawstring suture within the annular channel around the circumference of the split ring, tightening the drawstring suture to increase or maintain the compression force, and tying the drawstring suture.

7. The method of claim 2, wherein the providing step comprises providing a split ring formed of a biocompatible metal.

8. The method of claim 2, wherein the providing step comprises providing a garter spring formed of a biocompatible metal.

9. The method of claim 2, wherein the providing step comprises providing an O-ring formed of a biocompatible polymer.

10. The method of claim 2, wherein:
the providing step comprises providing a split ring formed of a biocompatible metal extending between a first split ring end pin receptacle and a second split ring end pin receptacle;
the expanding step comprises expanding the split ring to dispose the first and second split ring receptacles into operative alignment and fitting a locking member into the first and second split ring receptacles to maintain the second ring lumen diameter; and
the halting step comprises removing the locking member from the first and second split ring receptacles.

11. The method of claim 2, wherein:
the providing step comprises providing a split ring formed of a bio-compatible metal extending between a first loop at a first free end of the split ring and a second loop at a second free end of the split ring;
the expanding step comprises expanding the split ring to dispose the first and second loops into operative alignment and fitting a locking pin through the first and second loops to maintain the expanded ring lumen diameter; and
the halting step comprises removing the locking pin from the first and second loops.

12. The method of claim 2, wherein:
the providing step comprises providing a split ring formed of a bio-compatible metal extending between a first split ring end and a second split ring end; and
the expanding step comprises moving the first and second split ring ends apart to expand the ring lumen to the expanded ring lumen diameter.

13. The method of claim 2, wherein:
the providing step comprises providing a split ring formed of a bio-compatible metal extending between a first split ring end and a second split ring end; and
the expanding step comprises moving the first and second split ring ends apart as the ring lumen is expanded to the expanded ring lumen diameter.

14. The method of claim 2, further comprising the steps of:
wrapping an annular section of the graft proximal end around and over the compression ring disposing the graft proximal end against the graft sidewall; and
securing an edge of the graft proximal end to the graft sidewall.

15. The method of claim 2, wherein the providing step comprises attaching the compression ring around the graft proximal end.

16. The method of claim 15, wherein the providing step comprises forming the compression ring with a plurality of teeth extending inwardly into the graft lumen that may penetrate pores of the sewing ring.

17. The method of claim 15, wherein the providing step comprises forming the compression ring with a plurality of teeth extending inwardly into the graft lumen that may penetrate pores of the sewing ring.

18. The method of claim 1, wherein the prosthetic heart valve comprises one of a tissue valve and a mechanical valve.

19. The method of claim 1, wherein the graft sidewall is formed of a bio-compatible fabric sealed with a sealing material that inhibits loss of blood through fabric pores; and the prosthetic heart valve comprises a tissue valve packaged in a container with a preservative that preserves the tissue and that would attack the sealing material if the graft was coupled to the tissue valve and packaged in the container.

20. The method of claim 1, wherein the clamping step comprises:
fitting the graft proximal end against at least a portion of the annular exterior surface; and
applying a drawstring suture around the graft proximal end to compress the graft proximal end against the annular exterior surface of the prosthetic heart valve.

21. Apparatus for replacement of a section of blood vessel and a native heart valve comprising:
a prosthetic heart valve having an annular exterior surface;
an elongated vascular graft having a graft sidewall enclosing a vascular graft lumen and extending between graft proximal and distal ends; and
means for clamping the graft proximal end against at least a portion of the annular exterior surface without perforating the vascular graft to couple the vascular graft with the prosthetic heart during chronic implantation and to inhibit blood leakage through the proximal end of an elongated vascular graft.

22. The apparatus of claim 21, wherein said clamping means further comprises an expandable compression ring having a ring lumen adapted to be expanded from a first ring lumen diameter sized with respect to the annular exterior surface to a second ring lumen diameter enabling the fitting of the graft proximal end overlying the annular exterior surface within the expanded ring lumen, the compression ring adapted to apply compression force against the vascular graft proximal end and at least a portion of the annular exterior surface when the ring lumen diameter contracts from the second ring lumen diameter toward the first ring lumen diameter.

23. The apparatus of claim 22, wherein the prosthetic heart valve comprises a valve body supporting at least one occluding member and an annular, resilient sewing ring disposed about the valve body and the annular exterior surface has an annular surface diameter.

24. The apparatus of claim 23, wherein the compression ring is formed with a plurality of teeth extending inwardly into the graft lumen that may penetrate pores of the sewing ring.

25. The apparatus of claim 23, wherein the expandable compression ring has a first ring lumen diameter sized with respect to the annular surface diameter to provide an interference fit compressing the vascular graft proximal end against the resilient sewing ring.

26. The apparatus of claim 23, wherein the prosthetic heart valve comprises one of a tissue valve and a mechanical valve.

27. The apparatus of claim 22, wherein the compression ring comprises a split ring formed of a biocompatible metal and having an annular channel extending around the circumference of the split ring adapted to receive a drawstring suture tightened and tied to increase or maintain the compression force.

28. The apparatus of claim 22, wherein: the compression ring comprises:
a split ring formed of a biocompatible metal extending between a first split ring end pin receptacle and a second split ring end pin receptacle having a ring lumen adapted to be expanded from a first ring lumen diameter sized with respect to the annular exterior surface to the second ring lumen diameter enabling the fitting of the graft proximal end overlying the annular exterior surface within the expanded ring lumen; and
a locking member fitted into the first and second split ring receptacles to maintain the second ring lumen diameter and adapted to be removed to enable the contraction of the ring lumen diameter from the second ring lumen diameter toward the first ring lumen diameter to apply compression force against the vascular graft proximal end and at least a portion of the annular exterior surface.

29. The apparatus of claim 22, wherein the compression ring comprises a split ring formed of a biocompatible metal.

30. The apparatus of claim 22, wherein the compression ring comprises a garter spring formed of a biocompatible metal.

31. The apparatus of claim 22, wherein the compression ring comprises an O-ring formed of a biocompatible polymer.

32. The apparatus of claim 22, wherein:
the compression ring comprises a split ring formed of a bio-compatible metal extending between a first loop at a first free end of the split ring and a second loop at a second free end of the split ring having a ring lumen adapted to be expanded from a first ring lumen diameter sized with respect to the annular exterior surface to the second ring lumen diameter enabling the fitting of the graft proximal end overlying the annular exterior surface within the expanded ring lumen; and
a locking pin fitted into the first and second loops to maintain the second ring lumen diameter and adapted to be removed to enable the contraction of the ring lumen diameter from the second ring lumen diameter toward the first ring lumen diameter to apply compression force against the vascular graft proximal end and at least a portion of the annular exterior surface.

33. The apparatus of claim 22, wherein the compression ring comprises a split ring formed of a bio-compatible metal extending between a first split ring end and a second split ring end adapted to be engaged and moved apart to expand the ring lumen to the expanded ring lumen diameter.

34. The apparatus of claim 22, wherein the compression ring comprises a split ring formed of a bio-compatible metal extending between a first split ring end and a second split ring end, the first and second split ring ends movable apart as the ring lumen is expanded to the expanded ring lumen diameter.

35. The apparatus of claim 21, wherein the prosthetic heart valve comprises one of a tissue valve and a mechanical valve.

36. The apparatus of claim 21, wherein said clamping means further comprises an expandable compression ring fixedly attached to the graft proximal end having a ring lumen adapted to be expanded from a first ring lumen diameter sized with respect to the annular exterior surface to a second ring lumen diameter enabling the fitting of the graft proximal end overlying the annular exterior surface within the expanded ring lumen, the compression ring adapted to apply compression force against the vascular graft proximal end and at least a portion of the annular exterior surface when the ring lumen diameter contracts from the second ring lumen diameter toward the first ring lumen diameter.

37. The apparatus of claim 35, wherein the expandable compression ring further comprises a plurality of teeth extending inwardly into the graft lumen that may penetrate pores of the sewing ring.

38. The apparatus of claim 21, wherein the graft sidewall is formed of a bio-compatible fabric sealed with a sealing material that inhibits loss of blood through fabric pores; and the prosthetic heart valve comprises a tissue valve packaged in a container with a preservative that preserves the tissue and that would attack the sealing material if the graft was coupled to the tissue valve and packaged in the container.

39. The apparatus of claim 21, wherein the clamping means comprises a slip ring having a slip ring lumen sized with respect to the annular exterior surface enabling the fitting of the graft proximal end overlying the annular exterior surface within the slip ring lumen, whereby a drawstring suture can be tied adjacent the slip ring to apply compression force against the vascular graft proximal end adjacent the solid ring and at least a portion of the annular exterior surface when the drawstring suture is drawn tight.

40. The apparatus of claim 21, wherein the graft is formed having a proximal annular section having a radially expandable graft sidewall and a distal annular section having a longitudinally extendable graft sidewall.

41. Apparatus for replacement of a section of blood vessel and a native heart valve comprising:
a prosthetic heart valve having an annular exterior surface;
an elongated vascular graft having a graft sidewall enclosing a vascular graft lumen and extending between graft proximal and distal ends; and
an expandable compression ring having a ring lumen adapted to be expanded from a first ring lumen diameter sized with respect to the annular exterior surface to a second ring lumen diameter enabling the fitting of the graft proximal end overlying the annular exterior surface within the expanded ring lumen, the compression ring adapted to apply compression force against the vascular graft proximal end and at least a portion of the annular exterior surface when the ring lumen diameter contracts from the second ring lumen diameter toward the first ring lumen diameter.

42. The apparatus of claim 41, wherein the prosthetic heart valve comprises a valve body supporting at least one occluding member and an annular, resilient sewing ring disposed about the valve body and the annular exterior surface has an annular surface diameter.

43. The apparatus of claim 42, wherein the providing step comprises forming the compression ring with a plurality of teeth extending inwardly into the graft lumen that may penetrate pores of the sewing ring.

44. The apparatus of claim 42, wherein the compression ring has a first ring lumen diameter sized with respect to the annular surface diameter to provide an interference fit compressing the vascular graft proximal end against the resilient sewing ring.

45. The apparatus of claim 42, wherein the prosthetic heart valve comprises one of a tissue valve and a mechanical valve.

46. The apparatus of claim 41, wherein the compression ring comprises a split ring formed of a biocompatible metal.

47. The apparatus of claim 41, wherein the compression ring comprises a garter spring formed of a biocompatible metal.

48. The apparatus of claim 41, wherein the compression ring comprises an O-ring formed of a biocompatible polymer.

49. The apparatus of claim 41, wherein the compression ring comprises a split ring formed of a biocompatible metal and having an annular channel extending around the circumference of the split ring adapted to receive a drawstring suture tightened and tied to increase or maintain the compression force.

50. The apparatus of claim 41, wherein the compression ring comprises:
a split ring formed of a biocompatible metal extending between a first split ring end pin receptacle and a second split ring end pin receptacle having a ring lumen adapted to be expanded from a first ring lumen diameter sized with respect to the annular exterior surface to the second ring lumen diameter enabling the fitting of the graft proximal end overlying the annular exterior surface within the expanded ring lumen; and further comprising:
a locking member fitted into the first and second split ring receptacles to maintain the second ring lumen diameter and adapted to be removed to enable the contraction of the ring lumen diameter from the second ring lumen diameter toward the first ring lumen diameter to apply compression force against the vascular graft proximal end and at least a portion of the annular exterior surface.

51. The apparatus of claim 41, wherein the compression ring comprises:
a split ring formed of a bio-compatible metal extending between a first loop at a first free end of the split ring and a second loop at a second free end of the split ring having a ring lumen adapted to be expanded from a first ring lumen diameter sized with respect to the annular exterior surface to the second ring lumen diameter enabling the fitting of the graft proximal end overlying the annular exterior surface within the expanded ring lumen; and further comprising:
a locking pin fitted into the first and second loops to maintain the second ring lumen diameter and adapted to be removed to enable the contraction of the ring lumen diameter from the second ring lumen diameter toward the first ring lumen diameter to apply compression force against the vascular graft proximal end and at least a portion of the annular exterior surface.

52. The apparatus of claim 41, wherein the compression ring comprises a split ring formed of a bio-compatible metal extending between a first split ring end and a second split ring end adapted to be engaged and moved apart to expand the ring lumen to the expanded ring lumen diameter.

53. The apparatus of claim 41, wherein the compression ring comprises a split ring formed of a bio-compatible metal extending between a first split ring end and a second split ring end, the first and second split ring ends movable apart as the ring lumen is expanded to the expanded ring lumen diameter.

54. The apparatus of claim 41, wherein the prosthetic heart valve comprises one of a tissue valve and a mechanical valve.

55. The apparatus of claim 41, wherein said compression ring is fixedly attached to the graft proximal end.

56. The apparatus of claim 55, wherein the compression ring further comprises a plurality of teeth extending inwardly into the graft lumen that may penetrate pores of the sewing ring.

57. The apparatus of claim 41, wherein the graft sidewall is formed of a bio-compatible fabric sealed with a sealing material that inhibits loss of blood through fabric pores; and the prosthetic heart valve comprises a tissue valve packaged in a container with a preservative that preserves the tissue and that would attack the sealing material if the graft was coupled to the tissue valve and packaged in the container.

58. The apparatus of claim 41, wherein the graft is formed having a proximal annular section having a radially expandable graft sidewall and a distal annular section having a longitudinally extendable graft sidewall.

59. Apparatus for replacement of a section of blood vessel and a native heart valve comprising:
a prosthetic heart valve having an annular exterior surface;
an elongated vascular graft having a graft sidewall enclosing a vascular graft lumen and extending between graft proximal and distal ends, the graft distal end formed with a compression ring having a ring diameter adapted to fit over the annular exterior surface to enable application of a drawstring suture around the graft proximal end to compress the graft proximal end against the annular exterior surface of the prosthetic heart valve.

60. The apparatus of claim 59, wherein the compression ring is expandable from a first ring lumen diameter sized with respect to the annular exterior surface to a second ring lumen diameter enabling the fitting of the graft proximal end overlying the annular exterior surface within the expanded ring lumen, the compression ring adapted to apply compression force against the vascular graft proximal end and at least a portion of the annular exterior surface when the ring lumen diameter contracts from the second ring lumen diameter toward the first ring lumen diameter.

61. The apparatus of claim 59, wherein the compression ring is formed with a plurality of teeth extending inwardly into the graft lumen that may penetrate pores of the sewing ring.

62. The apparatus of claim 59, wherein the compression ring is a slip ring having a slip ring lumen sized with respect to the annular exterior surface enabling the fitting of the graft proximal end overlying the annular exterior surface within the ring lumen, whereby the drawstring suture applies compression force against the vascular graft proximal end adjacent the solid ring and at least a portion of the annular exterior surface when the drawstring is drawn tight.

63. The apparatus of claim 59, wherein the graft is formed having a proximal annular section having a radially expandable graft sidewall and a distal annular section having a longitudinally extendable graft sidewall.

64. Apparatus for replacement of a section of blood vessel and a native heart valve comprising:
a prosthetic heart valve having an annular exterior surface;
an elongated vascular graft having a graft sidewall enclosing a vascular graft lumen and extending between graft proximal and distal ends, the graft distal end adapted to fit over the annular exterior surface; and
a slip ring having a slip ring diameter sized with respect to the annular exterior surface enabling the fitting of the graft proximal end overlying the annular exterior surface within the slip ring lumen, to enable application of a drawstring suture around the graft proximal end to compress the graft proximal end adjacent the slip ring against the annular exterior surface of the prosthetic heart valve.

65. The apparatus of claim 64, wherein the slip ring is attached to the graft proximal end.

66. The apparatus of claim 59, wherein the graft is formed having a proximal annular section having a radially expandable graft sidewall and a distal annular section having a longitudinally extendable graft sidewall.
